(19) United States
(12) Patent Application Publication
(10) Pub. No.: US 2026/0090707 A1
(43) Pub. Date: Apr. 2, 2026

(54) INSTRUMENT SHAPE LOCKING

(71) Applicant: AURIS HEALTH, INC., Santa Clara, CA (US)

(72) Inventor: James Hayes, Redwood City, CA (US)

(73) Assignee: AURIS HEALTH, INC., Santa Clara, CA (US)

(21) Appl. No.: 18/904,058

(22) Filed: Oct. 1, 2024

(51) Int. Cl. A61B 1/005

(52) U.S. Cl. CPC A61B 1/0057

(57) ABSTRACT

An instrument includes a shaft, a first plurality of pull wire segments running a length of the shaft and terminating at a distal portion of the shaft, and a pull wire lock configured to lock tensions in the first plurality of pull wire segment. Navigating a medical instrument involves advancing a distal portion of an instrument from a distal end of a sheath, articulating the distal portion of the instrument to cause the distal portion of the instrument to assume a first shape, locking the distal portion of the instrument in the first shape, advancing a distal portion of the sheath over the distal portion of the instrument to cause the distal portion of the sheath to assume the first shape, and locking the distal portion of the sheath in the first shape.

### BACKGROUND

The invention relates generally to minimally-invasive instruments and systems, such as manually or robotically steerable catheter systems, and more particularly to steerable catheter systems for performing minimally invasive diagnostic and therapeutic procedures.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in an instrument having a shaft, a first plurality of pull wire segments running a length of the shaft and terminating at a distal portion of the shaft, and a pull wire lock configured to lock tensions in the first plurality of pull wire segments.

Another innovative aspect of the subject matter of this disclosure can be implemented in a system including a sheath having an elongate tube and a first plurality of pull wire segments running a length of the elongate tube and terminating at a distal portion of the elongate tube, an endoscope configured to be disposed within a lumen of the elongate tube of the sheath, a sheath pull wire lock configured to lock tensions in the first plurality of pull wire segments, and an endoscope pull wire lock. The endoscope includes a shaft and a second plurality of pull wire segments running a length of the shaft and terminating at a distal portion of the shaft, where the endoscope pull wire lock is configured to lock tension in the second plurality of pull wire segments.

Another innovative aspect of the subject matter of this disclosure can be implemented in a method of navigating a medical instrument including advancing a distal portion of an instrument from a distal end of a sheath, articulating the distal portion of the instrument to cause the distal portion of the instrument to assume a first shape, locking the distal portion of the instrument in the first shape, advancing a distal portion of the sheath over the distal portion of the instrument to cause the distal portion of the sheath to assume the first shape, and locking the distal portion of the sheath in the first shape.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates a robotic system arranged for a medical procedure in accordance with one or more embodiments.

FIG. 2 shows a robotically controllable sheath and endoscope assembly in accordance with one or more embodiments.

FIGS. 3A and 3B show schematic and cross-sectional views, respectively, of a flexible instrument having a plurality of articulation control pull wires in accordance with one or more embodiments.

FIGS. 4A and 4B show perspective and axial views, respectively, of an instrument including a plurality of pull wires in accordance with one or more embodiments.

FIGS. 5A and 5B show perspective and axial views, respectively, of an instrument including a braided jacket and a plurality of pull wires in accordance with one or more embodiments.

FIG. 6A shows a perspective view of a multi-segment instrument in accordance with one or more embodiments.

FIG. 6B-6E show axial views of the multi-segment instrument of FIG. 6A in accordance with one or more embodiments.

FIG. 7 is a schematic diagram of a multi-segment instrument in accordance with one or more embodiments.

FIG. 8 is a schematic diagram of the multi-segment instrument of FIG. 7 in a force isolated articulation state in accordance with one or more embodiments.

FIGS. 9A, 9B, 9C, and 9D provide a flow diagram illustrating a process for navigating instruments in accordance with one or more embodiments.

FIG. 10-16 show certain images corresponding to various blocks, states, and/or operations associated with the process of FIGS. 9A, 9B, 9C, and 9D in accordance with one or more embodiments.

### DETAILED DESCRIPTION

The headings provided herein are for convenience only and do not necessarily affect the scope or meaning of the claimed invention. Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Although certain spatially relative terms, such as “distal,” “proximal,” “outer,” “inner,” “upper,” “lower,” “below,” “above,” “vertical,” “horizontal,” “top,” “bottom,” “over,” “under,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that are similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

The present disclosure relates to systems, devices, and methods for locking the shape of articulable instruments, such as steerable endoscopes, catheters, sheaths, and other shaft-type instruments that utilize pull wires for instrument articulation. For example, instruments as described herein can be configured to assume a flexible, elastic configuration in which the pull wires thereof are free to translate to cause articulation of at least a portion of the instrument. With respect to catheters, access sheaths, endoscopes, robotic devices, and other medical devices relevant to the present disclosure, the term “instrument” is used according to its broad and ordinary meaning and may refer to any type of tool, device, assembly, system, apparatus, component, or the like. In some contexts herein, the term “device” may be used substantially interchangeably with the term “instrument.”

Aspects of the present disclosure relate to mechanisms for selectively locking the shape of an instrument by locking tension in pull wires associated with the instrument. For example, in accordance with aspects of the present disclosure, when the shaft of a steerable instrument has been articulated to a desired shape, the tension of the pull wires of the instrument can be locked/fixed, which can set the present shape of the instrument. If the pull wires are sufficiently axially stiff, such stiffness can prevent further articulation of the shaft. Instrument shape locking can allow for cooperative advancement/retraction of other instruments through and/or around the locked instrument without altering the shape of the locked instrument. In some implementations, an articulable instrument is configurable in a partially-locked state, wherein the articulation pull wires are held/secured, but allowed to slide/translate in response to force application with some amount of imposed friction. Such implementations can allow for adjustable pull wire friction, and can increase plasticity and/or reduce spring-back of the instrument shaft to a customizable degree.

Pull wires in connection with embodiments of the present disclosure can be contained at least partially within axially incompressible tubes that run a length of the instrument shaft. In some implementations, such tubes can be at least partially axially compressible. In some embodiments, pull wire tubes are integrated with fibers of a braided/woven jacket (e.g., tube) associated with the instrument. The term “fiber” is used herein according to its broad and ordinary meaning and may refer to any elongated piece of material, which may be spun into yarns, twisted into cords, braided to form strings, or otherwise configured. The term “fiber” can refer to any type of filament, thread, strand (e.g., bunch or assembly of filaments), cord, wire (e.g., metallic form), yarn, string, rope, ribbon, floss, or the like. “Fibers” effectively captures the variety of materials and constructions (such as twisted, braided, or woven) that can make up a braided jacket associated with an instrument (e.g., endoscope, sheath, or similar). The terms “associated” and “associated with” are used herein according to their broad and ordinary meanings. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

In some implementations, pull wires may terminate at one or more control plates (e.g., collar structures) associated with the instrument shaft. Such control plates can be positioned at a distal end of the shaft and/or at certain lengthwise positions along the shaft. In some embodiments, one or more control plates of an instrument may have one or more through-ports for pull wire(s) to pass through to a more-distal control plate. In some implementations, a braided jacket can be passed over the instrument pull wires and/or the associated pull wire tubes, wherein the braided jacket may be attached/fixed to one or more control plates. For example, tubes/lumens, at least some of which may be used to house pull wires, may be interwoven with the fibers of the braided jacket.

### Robotic Instrument Control and Medical Procedures

Disclosed herein are certain robotic systems that can be employed to perform a variety of medical procedures, such as endoscopic and laparoscopic procedures. During certain procedures, medical instrument(s), such as robotically controlled medical instrument(s) (e.g., endoscopes, access sheaths, working instruments), is/are inserted into a subject (e.g., a patient's body) and articulated or otherwise controlled, such as for the purpose of navigating to a target site. Within the subject, the instrument(s) may be positioned within a luminal network or other anatomy of the patient. As used herein, the term “luminal network” refers to any cavity structure within the body, whether comprising a plurality of lumens or branches (e.g., a plurality of branched lumens, as in the lungs or blood vessels) or a single lumen or branch (e.g., within the gastrointestinal tract).

Although certain aspects of the present disclosure are described in detail herein in the context of endoscopy procedures, such as bronchoscopy procedures, it should be understood that such context is provided for convenience and clarity, and instrument navigation concepts disclosed herein are applicable to any suitable medical procedures, such as various types of robotic medical procedures. Although certain principles disclosed herein are particularly applicable to the anatomy of the lungs and respiratory system, it should be understood that robotic instrument locking features disclosed herein may be implemented in connection with any suitable or desirable type of procedure, such as robotic bronchoscopy, laproscopy, arthroscopy, colonoscopy, laryngoscopy, neuroendoscopy, proctoscopy, anoscopy, gastroscopy, sigmoidoscopy, thoracoscopy, colposcopy, esophagoscopy, or other endoscopic or elongate-shaft-based procedure. It should be understood that any methodology described can be used in other contexts, such as animals, simulators, models of anatomy, cadavers, etc.

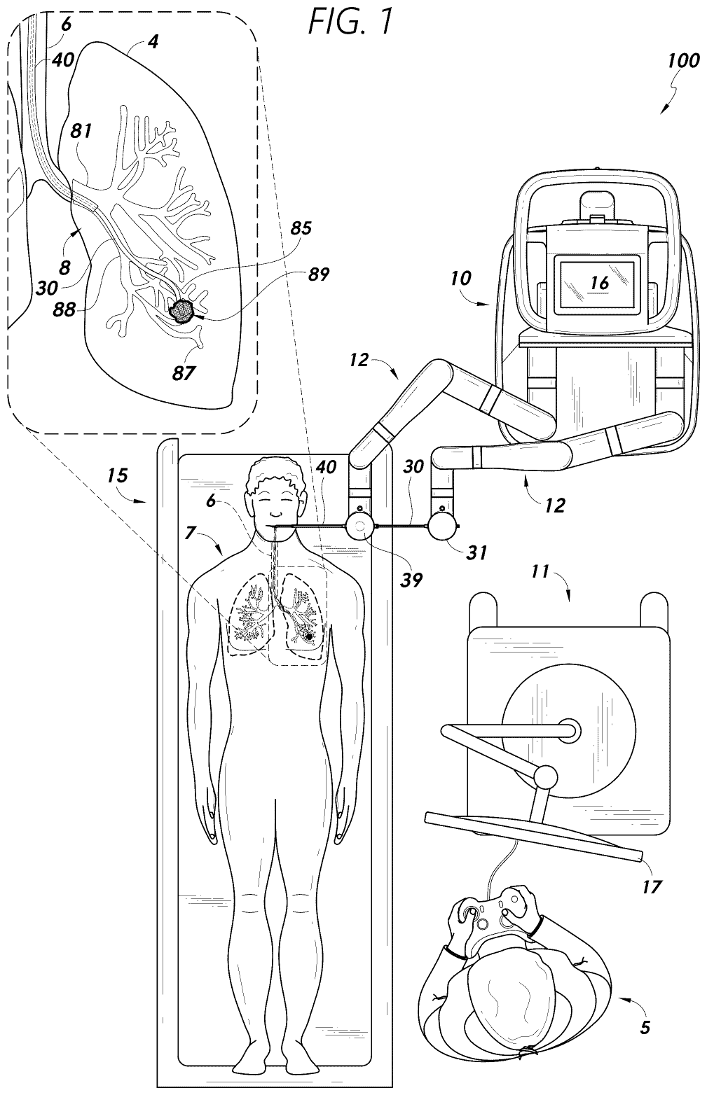

FIG. 1 illustrates an example medical system 100 for performing various medical procedures in accordance with aspects of the present disclosure. The medical system 100 may be used for, for example, endoscopic procedures. Robotic medical solutions can provide relatively higher precision, superior control, and/or superior hand-eye coordination with respect to certain instruments compared to strictly manual procedures. For example, robotic-assisted endoscopic access to patient anatomy can advantageously enable an operator to articulate an endoscope, sheath, or other instrument, using robotically-controlled gears/drives coupled to a handle/base portion of the instrument. The terms “endoscope” and “scope” are used herein according to their broad and ordinary meanings, and may refer to any type of elongate (e.g., shaft-type) medical instrument having image generating, viewing, and/or capturing functionality and being configured to be introduced into any type of organ, cavity, lumen, chamber, or space of a body/subject.

The medical system 100 includes a robotic system 10 (e.g., mobile robotic cart) configured to engage with and/or control a first instrument 30 (e.g., endoscope), including a proximal base 31, and a second instrument 40 (e.g., sheath), including a proximal base 39. The instrument 30 is shown configured to perform a direct-entry procedure on a patient 7. The term “direct-entry” is used herein according to its broad and ordinary meaning and may refer to any entry of instrumentation through a natural or artificial opening in a patient's body, such as the mouth. It should be understood that the instrument 30 and/or the instrument 40 may be any type of shaft-based medical instrument, including an endoscope (such as a bronchoscope), catheter (such as a steerable or non-steerable catheter), ureteroscope, nephroscope, gastroscope, laparoscope, or other type of medical instrument. Although direct entry of the instrument 30 is shown, aspects of the present disclosure relate to other types of subject entry, such as percutaneous entry.

The medical system 100 includes a control system 11 configured to interface with the robotic system 10, provide information regarding a procedure, and/or perform a variety of other operations. For example, the control system 11 can include one or more display(s) 17 configured to present certain information to assist the physician 5 and/or other technician(s) or individual(s). The medical system 100 can include a table 15 configured to hold the patient 7. Although the robotic arms 12 are shown in certain positions and coupled to certain instruments, it should be understood that such configurations are shown for convenience and illustration purposes, and the robotic arms may have different configurations over time and/or at different points during a medical procedure. Furthermore, the robotic arms 12 may be coupled to different devices/instruments than shown in FIG. 1, and in some cases or periods of time, one or more of the arms 12 may not be utilized or coupled to a medical instrument.

Articulation of one or both of the instruments 30, 40 may be controlled robotically, such as through operation of robotic manipulators associated with the robot arm(s) 12, wherein such operation may be controlled by the control system 11 and/or robotic system 10. The term “robotic manipulator” is used herein according to its broad and ordinary meaning and may refer to any type or configuration of one or more robotic end effectors, actuators, gears, drives, rails, interfaces, or the like.

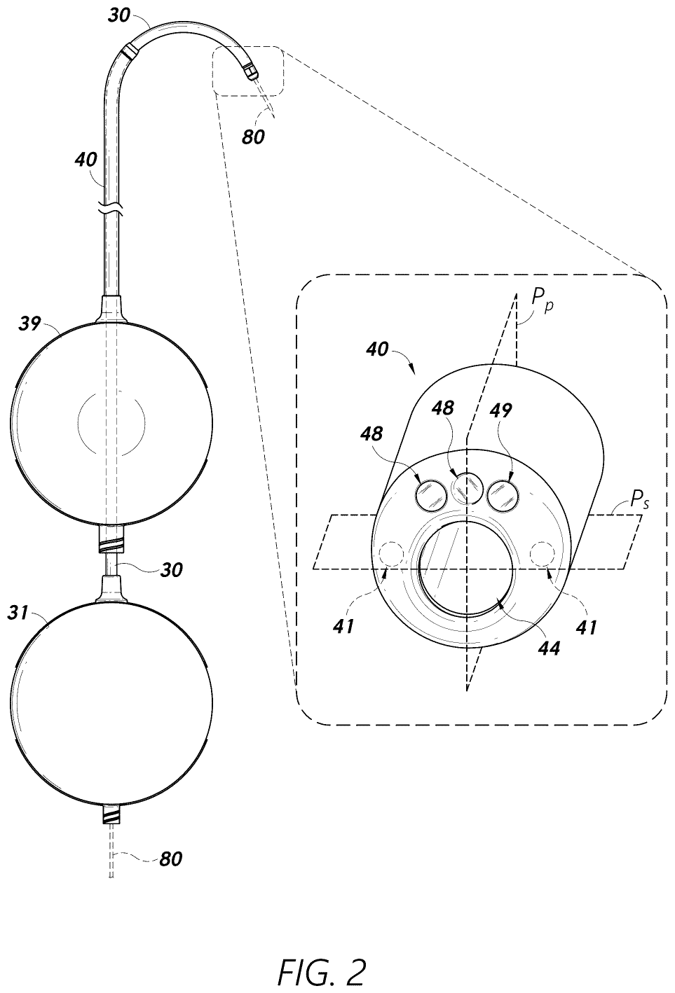

For example illustration purposes, FIG. 1 shows the robotic system 100 arranged for diagnostic and/or therapeutic bronchoscopy. During a bronchoscopy, the arm(s) 12 of the robotic system 10 may be configured to drive one or both of the instruments 30, 40 through a natural orifice access point (e.g., the mouth of the patient 7 positioned on a table 15 in the present example) to deliver diagnostic and/or therapeutic tools. As shown, the robotic system 10 (e.g., cart) may be positioned proximate to the patient's upper torso in order to provide access to the access point. The arrangement in FIG. 2 may also be utilized when performing an upper gastro-intestinal (GI) procedure with a gastroscope, a specialized endoscope for GI procedures. The robotic system 10 can include a display 16 for providing procedure-related information to the user.

With further reference to FIG. 1, the instrument 30 may be directed down the patient's trachea 6 and lungs after insertion using precise articulation commands from the robotic system 10 until reaching the target operative site. For example, the instrument 30 may be directed to deliver a biopsy needle to a target, such as, for example, a lesion or nodule 89 within the lungs of the patient 7. The needle may be deployed down a working channel that runs the length of the instrument 30 to obtain a tissue sample to be analyzed by a pathologist. In some instances, diagnostic and therapeutic treatments can be delivered in separate procedures.

For reference, FIG. 1 shows details of certain respiratory anatomy in which the instrument(s) 30, 40 may be advanced and/or articulated. Generally, the respiratory system comprises certain passages, vessels, organs, and muscles that aid the body in the exchange of gases between the air and blood, and between the blood and the cells of the body. The respiratory system includes the upper respiratory tract, which comprises the nose/nasal cavity, the pharynx (i.e., throat), and the larynx (i.e., voice box). The respiratory system further includes the lower respiratory tract, which is shown in detail and comprises the trachea 6, the lungs 4, and the various segments of the bronchial tree 8, including the alveoli and alveolar ducts, which comprise clusters of small air sacs that are responsible for gas exchange between the lungs and the pulmonary blood vessels. The bronchial tree 8 is an example luminal network in which robotically-controlled instruments may be navigated and articulated in accordance with the inventive solutions presented here. However, although aspects of the present disclosure are presented in the context of luminal networks including a bronchial network of airways (e.g., lumens, branches) of a patient's lung, embodiments of the present disclosure can be implemented in other types of luminal networks, such as renal networks, cardiovascular networks (e.g., arteries and veins), gastrointestinal tracts, urinary tracts, etc. The bronchial tree 8 includes primary bronchi 81, which branch off into smaller secondary 88 and tertiary 85 bronchi, and terminate in even smaller tubes called bronchioles 87. Each bronchiole tube is coupled to a cluster of alveoli.

Lung cancer and other cancers generally involve abnormal cell growth (e.g., in the area of the lungs or other anatomy), which can have the potential to invade or spread to other parts of the body. For example, cancer can form in tissues of the lung, such as in the cells that line the various air passages. When not treated in an effective and/or timely manner, lung cancers can spread/metastasize to lymph nodes or other organs in the body, which can severely impact patient recovery prospects. In FIG. 1, the patient 7 is shown having a mass of tissue 89, referred to as a lung nodule, that has formed in the area of the lungs 4. Such lung nodules can be benign or cancerous. Robotically-controlled instrumentation can be implemented to perform a diagnostic biopsy procedure from within the bronchial network to determine whether a lung nodule 89 is cancerous, or other treatment or therapy.

In the illustrated example, the instrument 30 is an endoscope and the instrument 40 is a sheath. The instrument 30 may be slidably positioned within a working channel/lumen of the instrument 40. The terms “lumen” and “channel” are used herein according to their broad and ordinary meanings and may refer to a physical structure forming a cavity, void, conduit, or other pathway, such as an at least partially rigid elongate tubular structure, or may refer to a cavity, void, pathway, or other channel, itself, that occupies a space within an elongate structure (e.g., a tubular structure). The telescopic arrangement of the instruments 30, 40 may allow for a relatively thin design of the instrument 30 and may improve a bend radius of the instrument 30 while providing a structural support via the instrument 40.

### Robotic Endoscope/sheath Navigation

FIG. 2 shows a robotically controllable sheath 40 and endoscope 30 assembly in accordance with one or more embodiments. The endoscope 30 can include a base 31 configured to be coupled to a robotic manipulator to facilitate robotic control/advancement of the endoscope 30. Another robotic manipulator may be coupled to a base 39 associated with the sheath 40 to facilitate advancement and/or articulation of the sheath 40. It should be understood that the instruments 30, 40 shown in FIG. 2 and described in connection therewith can be any type of medical instrument, such as any type of steerable sheath or catheter that may be utilized in connection with procedures/processes disclosed herein.

FIG. 2 includes a detailed image of the distal end of the endoscope 30. The endoscope 30 may include one or more working channels 44 through which additional instruments/tools, such as injection and/or biopsy needles, lithotripters, basketing devices, forceps, or the like, can be introduced into a treatment site. The endoscope 30 can be inserted through the lumen of the sheath 40 such that the endoscope 30 and/or sheath 40 can be controlled in a telescoping manner based on commands received from a user and/or automatically generated by the robotic system. In some implementations, a working channel instrument 80 (e.g., biopsy needle) may be coupled to a robotic manipulator, disposed within a working channel of the endoscope 30, and/or controlled in concert with the other instruments.

Each of the robotically controllable instruments may be articulable with a number of degrees of freedom. For example, an endoscope may be configured to move/articulate in multiple degrees of freedom, such as: insertion, roll, and articulation in various directions. In implementations in which an endoscope is manipulated within a controllable outer access sheath, the system may provide up to ten degrees of freedom, or more (e.g., for each instrument, the degrees of freedom may include: one insertion degree of freedom and four (or more) independent pull wires, each providing an independent articulation degree of freedom), which can allow for compound bending of the instrument.

The endoscope 30 can include one or more lights 49 and/or one or more cameras or other imaging devices 48. The endoscope 30 may further comprise one or more position sensors 41 (e.g., electromagnetic position sensors). The distal end of the endoscope 30 further includes an opening to the working channel 44. The endoscope 30 may further be configured to accommodate optical fibers to carry light from proximally located light sources, such as light-emitting diodes, to the distal end of the endoscope.

The various instruments disclosed herein, such as robotically articulable endoscopes and access sheaths, can have variable bending stiffness that facilitates advancement thereof through tortuous paths in a patient, including, but not limited to the pulmonary airways. In some embodiments, a variable bending stiffness medical instrument can include an elongated shaft that includes a plurality of sections having different bending stiffness properties. For example, a variable bending stiffness medical instrument can include a distal section that has a lower bending stiffness (e.g., that flexes or bends more easily and/or requires less force to flex or bend) than a bending stiffness of a proximal section. The distal section may be more easily bendable so as to facilitate steering or navigation of the medical instrument, while the proximal section can be relatively stiffer to facilitate pushing the medical instrument through an anatomical lumen.

Robotically controllable endoscopes in accordance with the present disclosure can be configured to provide relatively precise control near the distal tip/portion of the endoscope, which can be advantageous particularly after the endoscope has already been significantly bent or deflected to reach the desired target. The endoscope 30 and/or sheath 40 can be deflectable in one or two directions in each of two planes (e.g., Pₚ, Pₛ). One or more articulation control pull wires, which may have the form of any type of elongate cable, wire, tendon, or the like, can run along the outer surface of, and/or at least partially within, the shaft of the endoscope 30 and/or sheath 40. Any reference herein to a pull wire may be understood to refer to any segment of a pull wire. That is, description herein of pull wires can be understood to refer more generally to pull wire segments, which may comprise an entire wire end-to-end, or any length or subsegment thereof. The one or more pull wires of an articulable instrument described herein can include one, two, three, four, five, six or more pull wires or segments. Manipulation of the one or more pull wires can produce articulation of the articulation section of the associated instrument. Manipulation of the one or more pull wires can be controlled via one or more instrument drivers positioned within, or connected to, the instrument base. For example, the robotic attachment interface between the instrument base and the robotic manipulator can include one or more mechanical inputs (e.g., receptacles, pulleys, gears, spools), that are designed to be reciprocally mated with one or more torque couplers on an attachment surface of the robotic manipulator. Drive inputs associated with the instrument base can be configured to control or apply tension to the plurality of pull wires in response to drive outputs from the robotic manipulator. The pull wires may include any suitable or desirable materials, including any metallic and non-metallic materials such as stainless steel, Kevlar, tungsten, carbon fiber, and the like.

The endoscope 30 and/or sheath 40 can include an endoskeleton that is varied along its length to produce different bending stiffnesses in different regions of the shaft of the instrument. In some embodiments, the endoskeleton provides hoop strength and kink resistance for the shaft of the instrument. The endoskeleton can be formed, in some embodiments, of either nitinol or stainless steel, although other materials may also be used.

FIG. 2 shows the endoscope 30 positioned within the inner channel of the sheath 40. In some implementations, the endoscope 30 and the sheath 40 are independently controlled relative to one another. For example, a robotic manipulator coupled to the endoscope base 31 can move the endoscope base 31 to insert or retract the endoscope 30 relative to the sheath 40 and/or patient anatomy. Similarly, the robotic manipulator coupled to the sheath base 39 can move the sheath base 39 to insert or retract the sheath 40 relative to the endoscope 30 and/or patient anatomy. In some embodiments, only distal portions of the endoscope 30 and/or sheath 40 are articulable.

### Instrument Shape Locking

With respect to elongate medical instruments, such as steerable endoscopes and sheaths, generally the stiffness characteristics implemented in such instruments represent a trade-off between flexibility and pushability. For example, a more flexible instrument can generally allow for navigation of more torturous anatomy at the cost of increased risk or tendency to buckle in response to pushing/advancing of the instrument. Embodiments of the present disclosure advantageously provide for adjustable instrument shaft flexibility during a procedure, allowing for increased flexibility for tortuous navigation and selective stiffening of the instrument as needed to prevent undesirable deformation of the instrument in response to subsequent forces acting thereon.

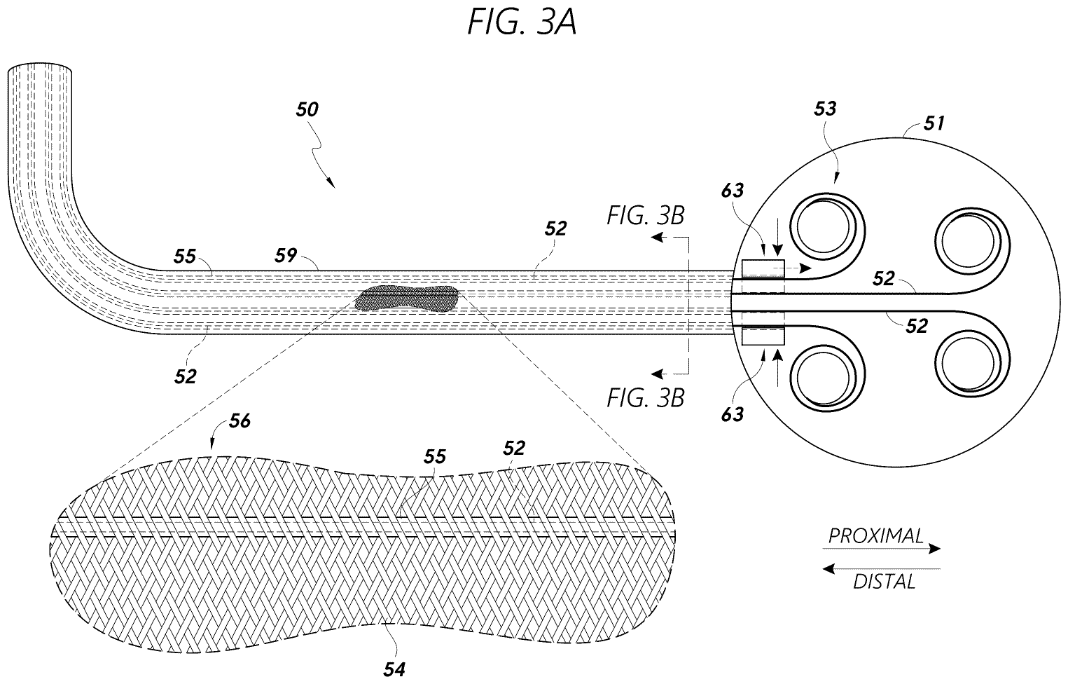

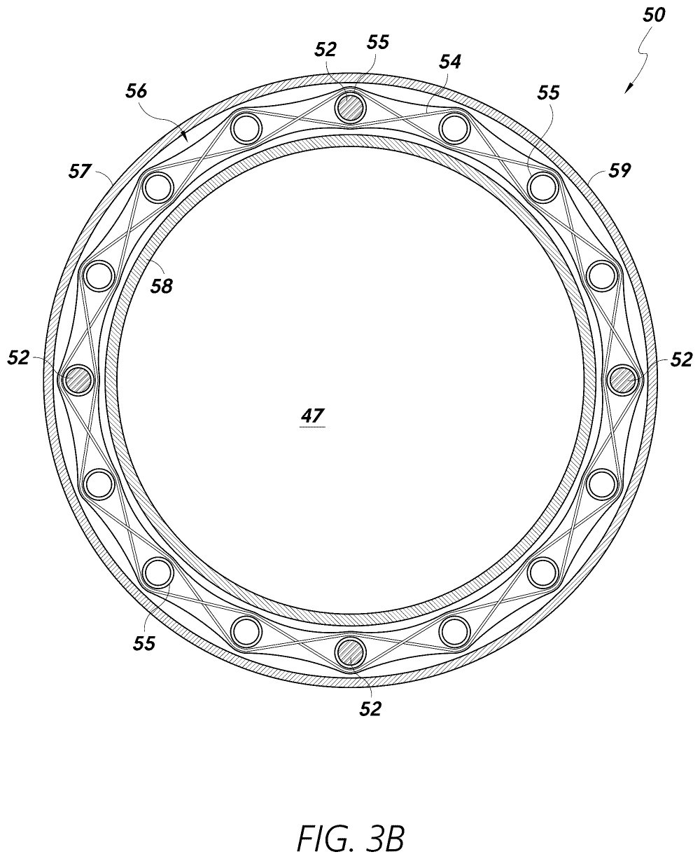

Instruments of the present disclosure can be configured to provide adjustable flexibility through the use of bend-and-stay features that provide dynamic instrument stiffness, wherein an instrument can be articulated to a desired shape and subsequently stiffened through pull wire tension locking to lock the present shape of the instrument. Instrument shape locking in connection with aspects of the present disclosure can allow for post-articulation engagement with the instrument, such as by passing another instrument over or through the articulated instrument, without undesirably deforming the articulated instrument. FIGS. 3A and 3B show schematic and cross-sectional views, respectively, of a flexible instrument 50 having a plurality of shape control pull wires 52, as well as one or more pull wire tension lock(s) 63 in accordance with one or more embodiments. The blocks identified as tension lock(s) 63 may represent a single lock configured to lock multiple or all pull wires, or may represent separate locks configured to lock a single respective pull wire.

The instrument 50 can include any number of pull wires 52, which may be interwoven or otherwise associated with a braided jacket 56 of the instrument 50. For example, the pull wire(s) 52 can pass through respective axially-incompressible tubes 55 that are interwoven with the fibers 54 of the braided jacket 56, as shown in the detailed close-up image in FIG. 3A. The braided fibers 54 can comprise steel fiber, tungsten, polymer, or other material. The tube(s) 55 can comprise polytetrafluoroethylene, polyimide, or the like. Although described and illustrated in some contexts as a braided tube, it should be understood that the jacket 56 can comprise any type of tube, such as braided or non-braided tubes, including an extrusion with a plurality of lumens serving as the tubes 55, a link-based tube or shaft with wire capture features serving as the tubes 55, or a laser-cut hypotube with welded on metal tubes, for example.

The instrument 50 can be configurable to a flexible state/configuration in which the pull wires 52 are free/permitted to axially translate and have the tension thereof adjusted, thereby causing articulation of the instrument 50. In the flexible state/configuration, the tension lock(s) 63 are in an at least partially unlocked state. The instrument 50 is further configurable to a shape-locked state/configuration in which one or more of the pull wires 52 have a tension thereof locked/fixed by the tension lock(s) 63. In the shape-locked state/configuration, further articulation of the instrument 50 is impeded or prevented due to the locked tension in the pull wire(s) 52. In some implementations, a partially-locked state/configuration may be configured in which one or more of the pull wires 52 are held but allowed to slide in response to certain force with added friction on the pull wire(s) from the tension lock(s) 63. The tension lock(s) 63 can be configurable in any number of intermediate states/configurations between fully unlocked and fully locked/clamped, thereby allowing for the user to set a particularly desired behavior with respect to instrument plasticity/spring-back. The tension lock(s) 63 can be implemented as clamp(s) that press down on a portion of the pull wire(s) 52 to hold their tension, or can be implemented as locks on the tensioning actuator(s) 53 that arrest/lock the rotation or other tension-affecting actuation thereof. The tension lock(s) 63 and/or tensioning actuator(s) 53 can be disposed or otherwise incorporated in a base 51 of the instrument 50. The instrument may be transitioned between the various tension lock states using robotic control via a robotic manipulator coupled to or otherwise associated with the instrument base 51.

As illustrated, the instrument 50 can include a shaft 59 and a base 31. The base 31 can include one or more pulleys/spools or other tensioning actuators 53. The pull wires 52 can be wound around the pulleys/spools, such that rotation thereof can alter tension in the pull wires. Each tensioning actuator 53 can be robotically controlled/rotated with certain drive outputs, which can be configured to provide torque to cause each tensioning actuator 53 to rotate to increase or decrease tension in an associated one of the pull wires 52. The base 31 can employ any known mechanisms to convert a torque/force received from a rotational axis of a drive output to the rotational axis of the tensioning actuator(s) 53, which may or may not have parallel rotational axes.

Each of the pull wires 52 may be fixed to a tip/end of the shaft 59, or may be fixed at an intermediate lengthwise position along the shaft 59. For example, the pull wires 52 can couple to opposing and/or distributed portions of a control plate associated with the tip/end of the shaft 59 or intermediate position thereof. With such fixed pull wire coupling to the articulation control plate(s), the instrument 50 (e.g., tip) can be articulated based on rotation/actuation of the tensioning actuator(s) 53. The pull wires 52 may be controlled by single-wire actuators, wherein each of the pull wires 52 is controlled by an independently actuatable actuator, or two or more of the pull wires 52 may be controlled by a single actuator, such as through opposing winding of two or more pull wires about a single pulley.

FIG. 3B shows a cross-sectional view of the instrument 50, which may be an endoscope, sheath, or other elongate instrument. The instrument 50 comprises an inner layer 58 disposed radially inside of the braided jacket 56 and an outer layer 57 disposed radially outside of the braided jacket 56. The inner layer 58 and outer layer 57 can surround various other features that enable some of the functionality of the instrument 50. The inner layer 58 and/or outer layer 57 can comprise polymer tubes or wraps. In some implementations, the inner layer 58 can comprise an endoskeleton, which may be formed of a laser cut hypotube made from a metal, such as nitinol or stainless steel, for example. The inner layer 58 and/or outer layer 57 can comprise low-friction liners to promote sliding of the instrument 50 within and/or without the structure thereof. The inner layer 58 can define a volume that is usable as a working channel 47, as shown.

The braided jacket 56 can be disposed between the inner 58 and outer 57 layers. The braided jacket 56 can have interwoven therewith a plurality of tubes 55, at least some of which may be utilized to contain/house the tension control pull wire(s) 52. In some implementations, the braided jacket 56 can include a thermoplastic coating or filler material, which can be melted into a composite structure with the braided fibers 54. The braided jacket 56 can provide mechanical structure and stability to the instrument 50. The mechanical properties of the braided jacket can be based on the durometer of the braid geometry, the braid pic count, and the braid angle. In general, lower durometer materials provide lower bending stiffness and higher durometer materials provide higher bending stiffness. Example materials that can be used as filler/coating for the braided jacket 56 include polyether block amide (e.g., Pebax), nylon, plastics, and the like. The braided jacket 56 can include a braid made from a thermoplastic material.

The instrument 50, as configured, can provide for elasticity characteristics that can be adjusted in real time through the manipulation of the tension lock(s) 63. In some implementations, each lockable pull wire can have a dedicated locking feature (e.g., clamp). Alternatively, two or more of the pull wires 52 may be lockable using a single tension lock 63. In some implementations, the tension lock(s) 63 can be controlled to add pull wire friction during dearticulation and/or to remove/decrease pull wire friction during articulation.

The tension lock(s) 63 can be implemented as toggle clamps. For example, the depiction of blocks as the tension lock(s) 63 can indicate a mechanism to pinch down on the various pull wires 52 in a manner as to lock the tension, and therefore shape, thereof. In some implementations, the tension lock(s) 63 are configured to clamp against the pull wires 52 in a dimension normal to the surface of the pull wires (e.g., perpendicular to the axis of the pull wire), as well as slide in the proximal direction to thereby pull the pull wire(s) proximally to some degree to provide additional stiffness/tension.

In some implementations, shape locking of the shaft 59 can be implemented using a vacuum pressure mechanism. However, such implementations may require larger shaft diameter to accommodate multiple walls and/or a central spine that provides a sufficient mechanical structure. Thus, such implementations can have certain limits in miniaturization associated therewith that are not present with other embodiments of the present disclosure that require only a single wall.

The configuration of the pull wires 52 contained in tubes 55 and interwoven with the braided jacket 56 can allow for reduced wall thickness in the instrument shaft 59. The braided fibers 54 of the braided jacket 56 can serve as a structural element to hold the pull wire tubes 55 in place.

The instrument 50 can be any type of steerable catheter, such as a steerable sheath comprising a working channel 47 for providing access to a part of a subject's anatomy, wherein any type of device may be insertable in the working channel 47 of the instrument. In addition to the working channel 47, which may be coaxial with the shaft 59 as shown or offset as in other examples disclosed herein (see, e.g., FIG. 2), one or more of the interwoven tubes 55 may be used for working instrument passage. For example, while one or more of the tubes 55 may be utilized for pull wire containment/housing, any otherwise unoccupied/empty ones of the tubes 55 can be used for working instrument(s), such as fiber optic cables, needles, etc.

The braid fibers 54 can be arranged in a helical pattern in overlapping directions along the length of the instrument 50. In some implementations, thirty-two (or other number) strands of fibers 54 are arranged in a helical pattern to form the braid of the braided jacket 56, with half in one winding direction/chirality and half in an opposing winding direction/chirality. In forming the braided jacket 56, braiding/fibers may be laid down over one or more process mandrels. The tubes 55 can advantageously be physically intertwined with the braided jacket 56, becoming a part of the braided jacket's structure. The tubes 55 are held in place by the fibers 54 of the braided jacket 56.

The instrument 50 of FIGS. 3A and 3B advantageously provides a minimally-invasive, flexible tool configured to navigate through complex torturous anatomy with reduced risk of prolapse/buckling, which can be particularly useful in navigation of the lungs or lower intestines. The following description and related figures relate to instruments having features similar to at least some of the features of the instrument 50 described above.

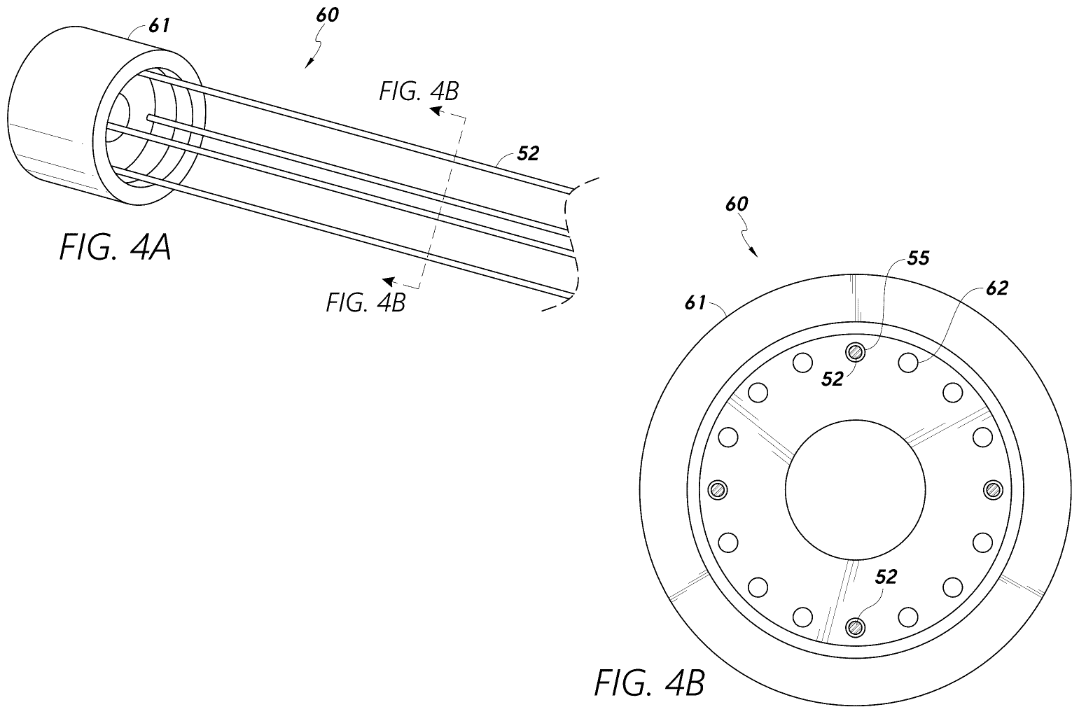

FIGS. 4A and 4B show perspective and axial views, respectively, of an instrument 60 including a plurality of pull wires 52 in accordance with one or more embodiments. Although the pull wires are shown in some figures of the present disclosure without respective tubes disposed thereabout, it should be understood that such presentations are in the interest of clarity, and any pull wire shown in a figure of the present disclosure may have a pull wire tube in which the pull wire is disposed.

The instrument 60 is shown in FIGS. 4A and 4B as not including a full shaft assembly, but rather merely pull wires 52 and/or pull wire tubes 55 terminating at a control plate 61, which may be associated with a distal tip of the instrument 60 and may have a collar structure. In the illustrated example, the control plate 61 has four pull wires 52 attached/fixed thereto. It may be advantageous to implement the instrument 60 with a minimum of three pull wires terminating at attachment with the control plate 61 to provide more than two degrees of freedom in articulation at the control plate 61. In some embodiments, the pull wires 52 and/or associated tubes 55 may be fixed to respective ports 62 in the control plate 61. Additional ports 62 may further be implemented in the control plate 61, which may be unused by pull wires, but may provide optional ports for other tubes, wires, devices, etc.

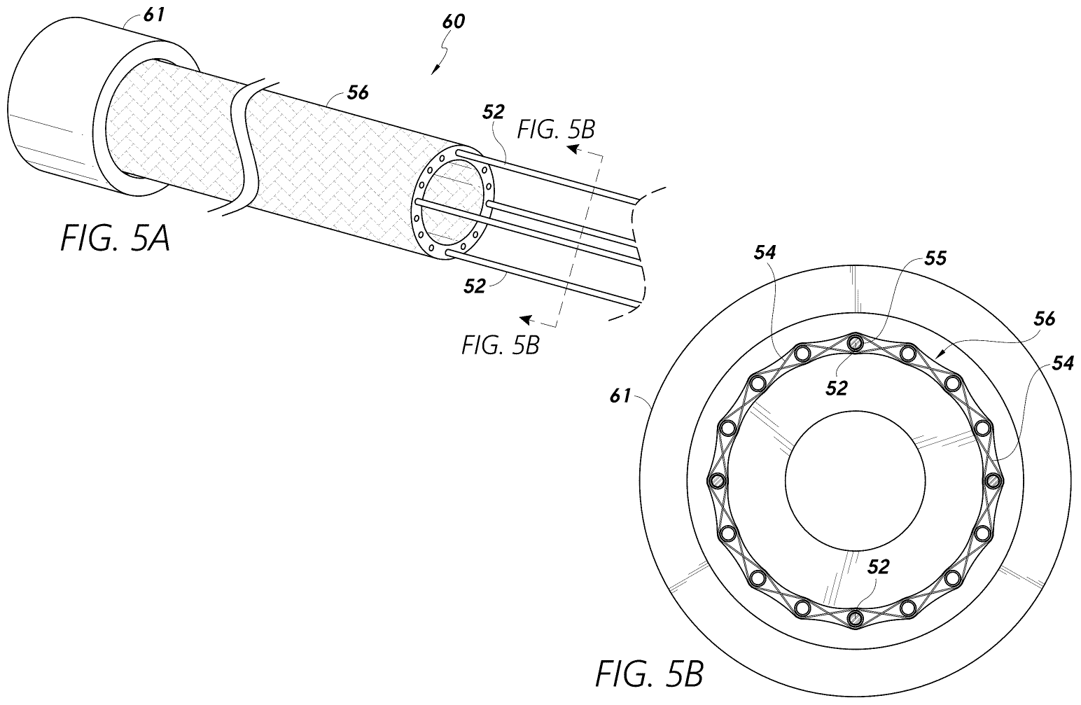

FIGS. 5A and 5B show perspective and axial views, respectively, of the instrument 60 including a braided jacket 56 having the plurality of pull wires 52 and/or associated tubes 55 interwoven therewith in accordance with one or more embodiments. In an assembly process, the braided jacket 56 can be passed over the pull wires 52 and/or tubes 55 and bonded to the control plate 61. In some implementations, a 32-wire braid may be used, which may advantageously allow for interweaving of up to sixteen wires/tubes, or more. The combination of the pull wire tubes 55, pull wires 52, and braided jacket 56 can be considered a shaft assembly or shaft segment. The cross-sectional view of FIG. 5B shows the tubes 55 interwoven between fibers 54 of the braided jacket 56. As shown, a subset of the total number of tubes 55 may be used for holding pull wires 52, whereas at least some of the tubes 55 may be open or otherwise occupied in the area of the instrument 60 shown in FIG. 5A.

Once the pull wires 52 and/or tubes 55 are interwoven with the braided jacket 56, one or more outer polymer tubes (not shown for visual clarity) can be slid over the outer diameter of the braided jacket 56 to form the shaft assembly. The assembly can be heated to melt/flow the polymer over the braided jacket 56. The proximal end of the instrument 60 can be mounted to a base, and the proximal ends/portions of the pull wires 52 can be installed on tensioning actuators (e.g., spools/pulleys) of the instrument base. The outer tubes/layers around the braided jacket 56 can include an outer polymer tube/layer that is melted/flowed, as well as heat shrink tubing that serves to shrink and compresses the melted polymer tube/layer into the braid. The term “tube” is used herein according to its broad and ordinary meaning, and may refer to any type of shaft, liner, conduit, pipe, cylinder, duct, hose, sleeve, vessel, or the like.

FIG. 5A shows only a single control plate 61, which may be associated with a distal portion of the instrument 60, wherein the control plate 61 can be configured to control articulation of a single segment of the instrument 60 (e.g., distal articulable segment). It should be understood that any of the steerable instruments disclosed herein can be implemented with multiple articulation segments, each associated with a separate control plate fixed to one or more of the pull wires of the instrument. Such embodiments can resemble repeated/stacked assemblies like those shown in FIG. 5A.

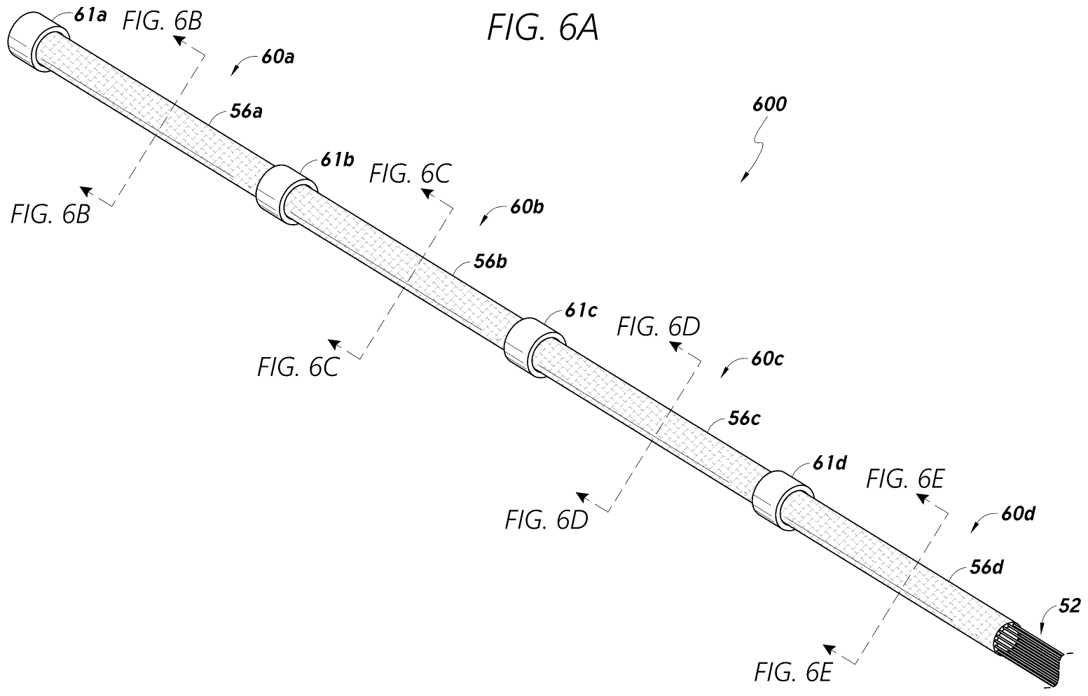

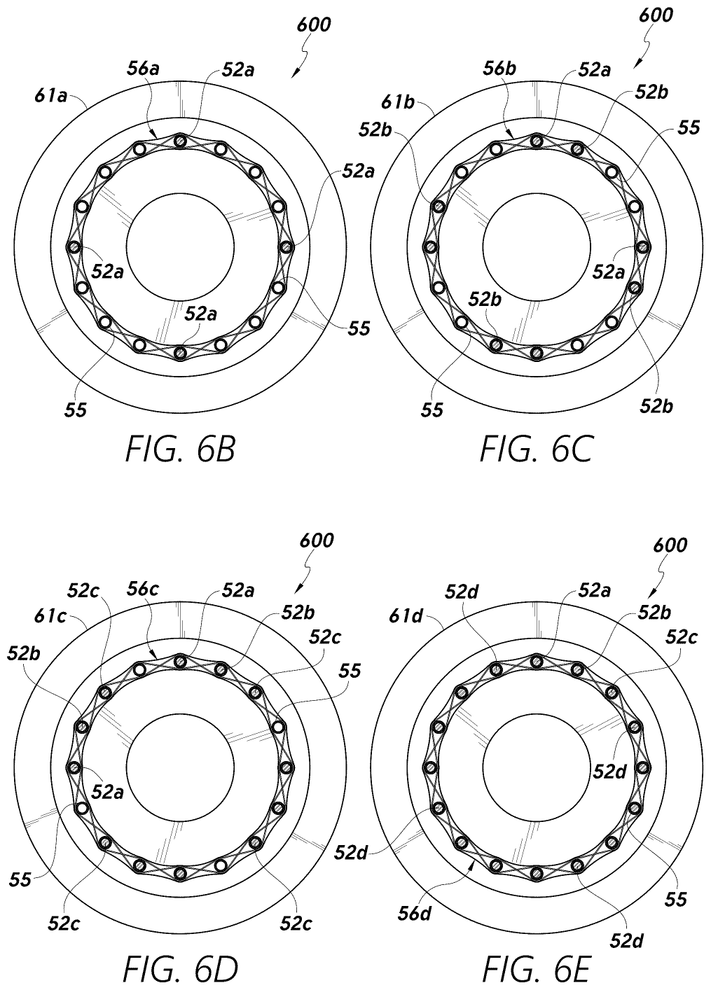

FIG. 6A shows a perspective view of a multi-segment instrument 600 in accordance with one or more embodiments. FIG. 6B-6E show axial views of the multi-segment instrument 600 of FIG. 6A in accordance with one or more embodiments. The shaft of the instrument 600 can house pull wires 52 that are fixed to and/or terminate at respective ones of a plurality of control plates 61, including a distal control plate 61a, as well as a plurality of intermediate control plates 61b, 61c, 61d. The control plates 61 can be considered collar structures in some implementations. The use of multiple control plates facilitates multiple articulating sections that can provide improved articulation complexity, possibly at the cost of requiring more drive motors for pull wire tension control.

The instrument 600 can be implemented in connection with any of the processes disclosed herein, wherein a user can robotically tension the pull wires to deform the instrument shaft to a desired shape, at which point the stiffness of the instrument can be increased and its shape set. Then a second instrument (e.g., endoscope or sheath) having similar pull wire tension locking features can be driven through or around the first instrument to a deeper navigation position. Once the second instrument can navigate no further, such as due to the risk of buckling, the stiffness of the second instrument can be increased through pull wire tension locking in accordance with aspects of the present disclosure, thereby locking its shape. The first instrument (e.g., sheath) may then be unlocked and made more flexible, by releasing or otherwise actuating the pull wire tension lock(s), and translated forward. As the first instrument translates forward, it may advantageously conform to the shape of the second instrument that had been made rigid. Once the first instrument reaches the tip of the second instrument, the first instrument (e.g., sheath) can be made stiff again and the second instrument can become flexible. This process could be repeated as needed to navigate deeper and deeper into torturous anatomy. In some implementations, one tensioning motor/actuator may be used for actuating the tension lock(s) for each of the first and second instruments. Additional motors can be used for controlling tensioning of the various articulation pull wires of the instruments.

The embodiment of FIG. 6A includes at least four lengthwise segments 60a, 60b, 60c, 60d (segments 60a-60d also referred to as ‘shaft assemblies’), each segment including a respective control plate 61a, 61b, 61c, 61d having a plurality of pull wires terminating thereat. The shaft segments 61a-61d can be considered shaft sections that each include one of the control plates 61 and a proximally-adjacent shaft and/or braid portion 56. Therefore, the instrument 600 can be considered to comprise multiple axially-offset shaft segments 60. Whereas embodiments providing only a single control plate may only be articulable to hold a single curvature, the embodiment of FIG. 6A can advantageously allow for articulation and locking of multiple separate segments, thereby allowing for compound bending of the instrument 600.

With the implementation of four stacked shaft segments/assemblies 60a-60d, each having a separate subset of articulation pull wires terminating at its respective control plate, four independent curvatures may be achievable using bend-and-stay locking features as described herein. Therefore, the instrument 600 may be bendable into a shape including two ‘S’ bends, with a curve inflection point between each segment. As more curvature complexity is desired, more shaft assemblies may be stacked and the length of each section (e.g., braided jacket sections 56a-56d) of the braided jacket 56 may be reduced. In various implementations, braid segments 56 having varying/different length may be implemented in a single instrument. For example, it may be desirable to have shorter distal segments/braids and longer proximal braids to enhance articulation closer to the tip of the instrument while favoring stiffness in a proximal portion of the instrument shaft. Generally, the more flexible the braid used in a given segment, the shorter it may be desirable for it to be to provide desirable stability. Stacking in this way may not necessitate more motors. For example, the instrument 600 may be implemented such that only a single motor/lock is used to hold all the lockable pull wires 52 in place to lock the shape of the instrument 600.

By providing selectable locking of the different shaft/pull-wire segments 60a-60d, the stacked instrument shaft of FIG. 6A can have the effect of a variable-length Bowden tube. For example, by locking a particular segment of the instrument 600, force isolation of the locked segment can act as a Bowden tube running the length of the locked segment. FIG. 6E shows a proximal-most control plate 61d, which has a plurality of ports therein. The ports may have tubes running therethrough, wherein at least some of the tubes contain one of the pull wires 52. In the particular embodiment shown, the control plate 61d has 16 ports, wherein each of the ports has a respective tube 55 associated therewith. Each of the tubes 55 may further have running therethrough a respective one of the pull wires 52. A subset 52d of the pull wires 52 may be fixed to the control plate 61d, wherein the remaining ones of the pull wires 52 may pass through ports of the control plate 61d without being fixed thereto, such that the passthrough pull wires can axially translate relative to the control plate 61d. Due to fixation with the control plate 61d, the tensioning of the pull wires 52d can cause articulation of the control plate 61d, such that the pull wires 52d can be used to articulate the instrument segment 60d.

FIG. 6D shows some of the pull wires that pass distally past the control plate 61d. The ports associated with the subset 52d of pull wires shown in FIG. 6D may not have wires passing therethrough in the region associated with the ports of the control plate 61c, as such pull wires may terminate at the more proximal control plate 61d. A subset of the remaining pull wires 52c may be fixed to the control plate 61c, such that the pull wires 52c may control articulation of the control plate 61c. Additional ones of the pull wires 52 may pass through the ports of the control plate 61c to more distal control plate(s)/segment(s) of the instrument 600. For example, FIG. 6C shows a view of the more distal control plate 61b and braided jacket segment 56b between the control plate 61c and the control plate 61b. Pull wires subset 52b passes through the control plate 61c to the control plate 61b, where such pull wires terminate. The remaining pull wires 52a pass through the control plate 61b to the distal control plate 61a, where they terminate and can be used to control articulation of the distal tip segment 60a of the instrument 600. As shown in FIG. 6B, the distal control plate 61a is coupled to the pull wires 52a, whereas the remaining ports of the control plate 61a may be unoccupied, as any pull wire segments associated therewith will have terminated at more proximal positions along the shaft of the instrument 600. Unoccupied port(s) in any of the control plates may have empty tubes 55 running therethrough.

As shown in FIG. 6A-6E, while some steerable instruments utilize only four or fewer articulation pull wires along the entire length thereof, some embodiments of the present disclosure advantageously include more than four articulation pull wires, such as eight, twelve, sixteen, or more. In such embodiments, separate subsets of the pull wires may be fixed to and/or terminate at separate control plates of the instrument. Each of the pull wires may be disposed at least partially within a dedicated tube 55 interwoven or otherwise associated with one or more of the braided jacket segments 56a, 56b, 56c, 56d. Tension-lockable pull wires that terminate at different ones of the control plates 61a-61d can provide force isolation, wherein forces can be transmitted to a certain segment while preventing movement elsewhere (i.e., unintended motion), wherein an active region of the instrument 600 is articulable, while the isolated region(s) is/are passive.

In the implementation of FIG. 6B-6E, the pull wire subsets 52a-52d each include four pull wires distributed evenly across four quadrants of the circular control plates. Each subset is offset from the others by one position along the circle's circumference. This offset creates a pattern where each subset begins one step clockwise (or counterclockwise) from the previous or subsequent subset's starting point, thus covering the entire circle uniformly and symmetrically. Each subset effectively marks every fourth port in a sequence that circles back to its start, ensuring that all points are equally spaced and aligned in a structured radial configuration around the center. Although a particular scheme of pull wire subsets and ports is shown, it should be understood that any configuration may be implemented.

It may be desirable to fix subsets of pull wires at articulation control plates that include at least three pull wires to be able to lock a shape of an associated segment of the instrument. For example, where only two pull wires are fixed to a given control plate and tension-locked, motion may be restricted only in the plane that intersects the two pull wires. Therefore, implementing control plate pull wire fixing of groups of three or four pull wires per plate, as described and shown herein, can provide locking in multiple dimensions.

As an example use case with respect to the embodiment of FIG. 6A, dynamically changing a driving modality may involve locking the shape of the three most proximal segments 60b, 60c, 60d, such as by tension-locking the pull wire subsets 52b, 52c, 52c, and using the unlocked subset 52a of pull wires to articulate the distal tip segment 60a in any direction. For example, each of the pull wires 52a can be tied to a motor and tensioned to create a force vector in the given direction. Where articulation in only one plane is desired, articulation in other plane(s) can be locked by clamping down on the opposing wires, thereby providing a relatively stable plane of articulation and less directional accuracy error.

In some implementations, one or more proximal subsets of pull wires (e.g., pull wire subsets 52b, 52c, 52d) may be locked using a single tension lock/motor. In such embodiments, articulation wires may only be connected to the distal most control plate 61a. For example, two or more of the remaining control plates 61b, 61c, 61d can be locked to a single motor, which can only apply tension uniformly.

By implementing the pull wire tubes 55 within the braid of the jackets 56, the outer diameter of the instrument 600 can be reduced without unduly sacrificing performance. A smaller instrument outer diameter can result in less stress in the shaft wall during articulation, and therefore less degradation over time. Furthermore, embodiments of the present disclosure may require relatively low articulation forces, allowing for the use of relatively smaller tendons, further reducing the profile/diameter of the instrument 600. More precise force control algorithms can also be enabled due to reduction in the signal-to-noise ratio, thus improving driving performance and collision sensitivity. Finally, a reduced instrument outer diameter/profile can allow for the device to navigate through tighter anatomy.

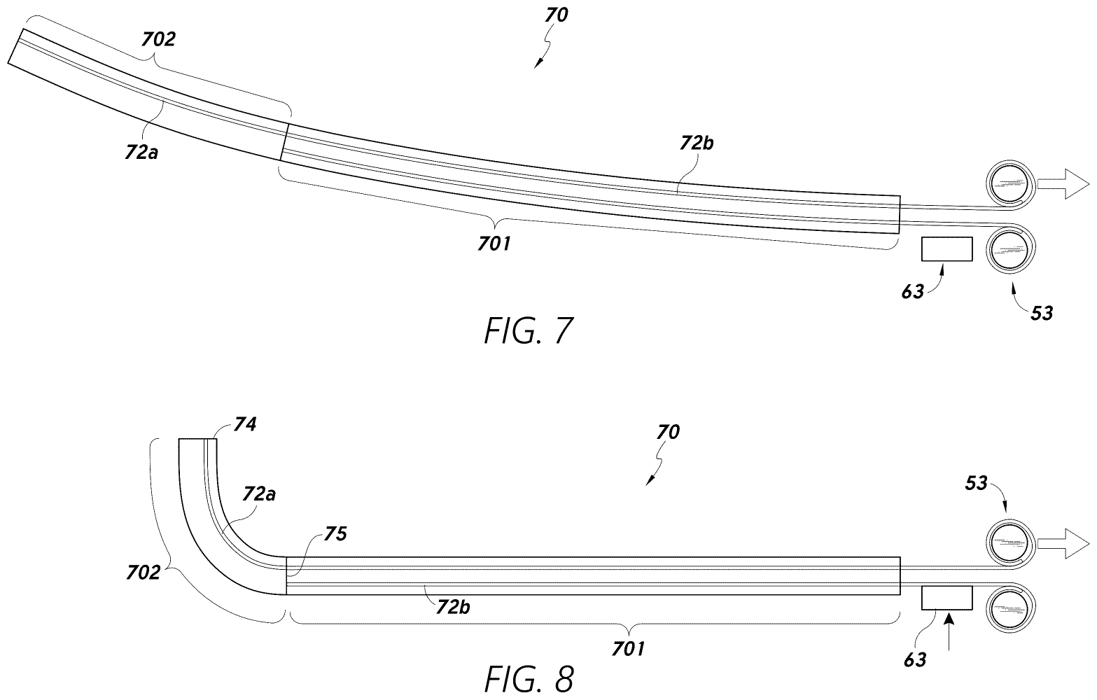

Aspects of instrument shape locking in accordance with the present disclosure can further be understood with reference to the diagrams of FIGS. 7 and 8. In particular, FIG. 7 is a schematic diagram of a multi-segment instrument 70 in accordance with one or more embodiments, wherein force isolation using pull wire tension locking as disclosed herein is not implemented. Alternatively, FIG. 8 shows articulation of the instrument 70 with force isolation implemented.

For clarity, only two pull wires are shown in the schematic diagrams of FIGS. 7 and 8, though the principles demonstrated therein apply to implementations using more than two pull wires. In particular, in the illustrated configuration, the instrument 70 includes a first pull wire 72a and a second pull wire 72b, wherein the first pull wire is fixed to and/or terminates at a distal end 74 of the instrument 70, whereas the second pull wire 72b is fixed to and/or terminates at an intermediate position 75 along the length of the instrument 70. The instrument 70 includes a tension lock 63 associated with at least the second pull wire 72b. In the configuration of FIG. 7, the tension lock 63 is in an unlocked state, in which the second pull wire 72b is free to be tensioned by an associated tensioning actuator 53. The tension lock 63 may be configured to lock one or both/all of the pull wires.

In the configuration of FIG. 7, wherein the tension lock 63 associated with the second pull wire 72b is not engaged, force isolation of the proximal segment 701 is not implemented. Therefore, tensioning of the first pull wire 72a without tensioning of the second pull wire 72b, as shown in FIG. 7, may cause upward bending/curving along the entire length of the instrument 70. Alternatively, if the second pull wire 72b is tensioned without tensioning of the first pull wire 72a, the instrument 70 may bend/curve downward up to the termination point at the intermediate position 75 of the second pull wire 72b, but not beyond such point.

In the configuration of FIG. 8, wherein the tension lock 63 associated with the second pull wire 72b is engaged, force isolation of the proximal segment 701 is implemented in opposition to upward articulation of the instrument 70 from tensioning of the first pull wire 72a. Therefore, tensioning of the first pull wire 72a, as shown in FIG. 8, may cause upward bending/curving along the distal segment 702, but not in the locked proximal segment 701. For example, the upward articulation force of the first pull wire 72a in the proximal segment 701 may be canceled by the opposing force of the second pull wire 72b caused by the tension locking thereof. With the ability to selectively lock the shape of the proximal segment 701, the instrument 70 can advantageously allow for articulation of the entire shaft of the instrument 70, or alternatively, force-isolated articulation of only the distal segment 702, as in FIG. 8.

Certain of the pull wire tension locking features described herein can advantageously transition from locked to unlocked states/configurations relatively quickly compared to other solutions that that may utilize magnets or shape memory materials for locking shape, which may not be as reactive as, for example, pull wire clamps as described herein. Furthermore, when in the unlocked state, the instrument shaft may advantageously remain highly flexible compared to certain other solutions (e.g., goose neck mechanisms).

Embodiments of the present disclosure can provide buckling protection without the need for a relatively stiff shaft to avoid sheath buckling. For example, desired stiffness can come from locked pull wires controlling a series of bends rather than stiff shaft tubes. Thus, the balance of stiffness to pushability can be avoided to some degree. Embodiments of the present disclosure can further allow for the robotic controlling/implementation of an arbitrary number of curvatures without requiring additional motors. For example, in some implementations, only one motor may be required/used to lock all of the pull wire segments. By implementing pull wire tension locking in connection with embodiments of the present disclosure, the need for anatomy to hold the desired shape of an instrument may be obviated.

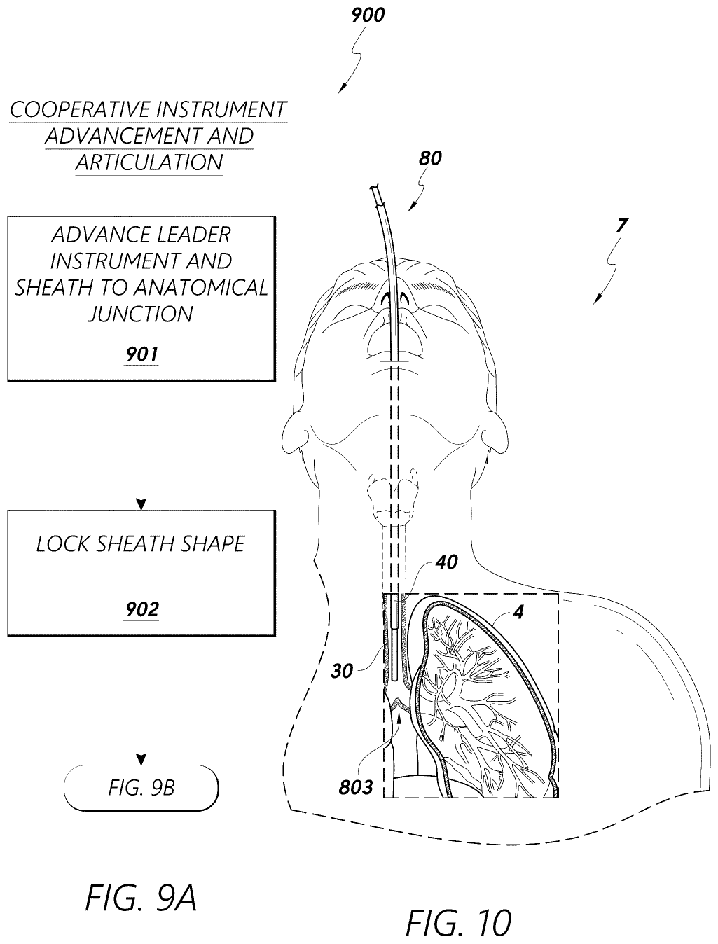

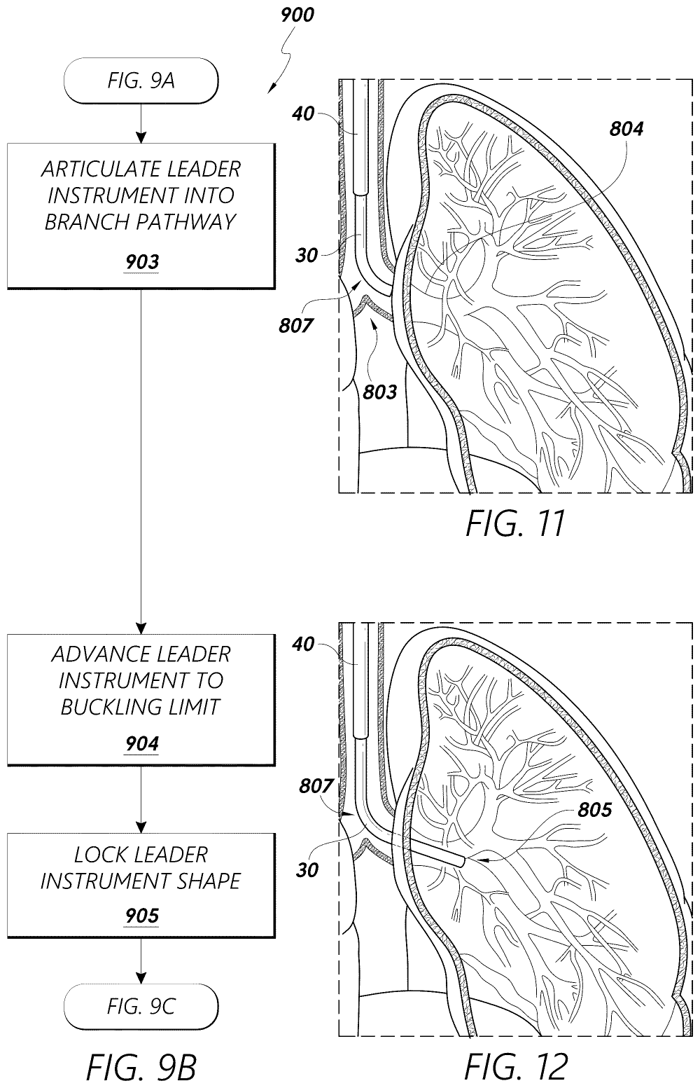

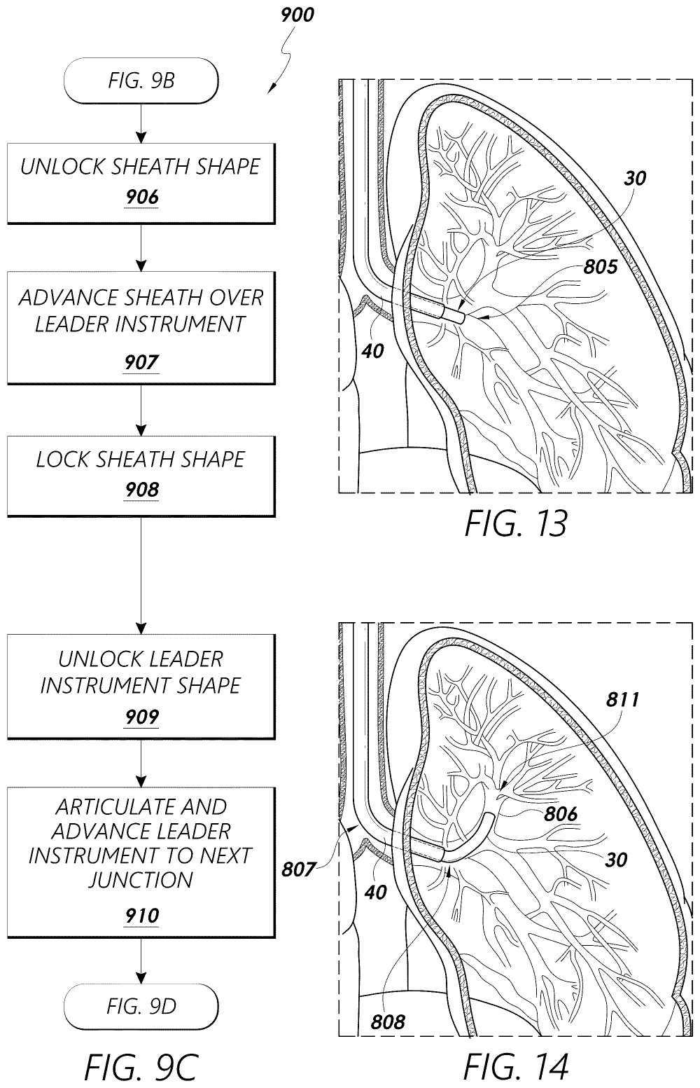

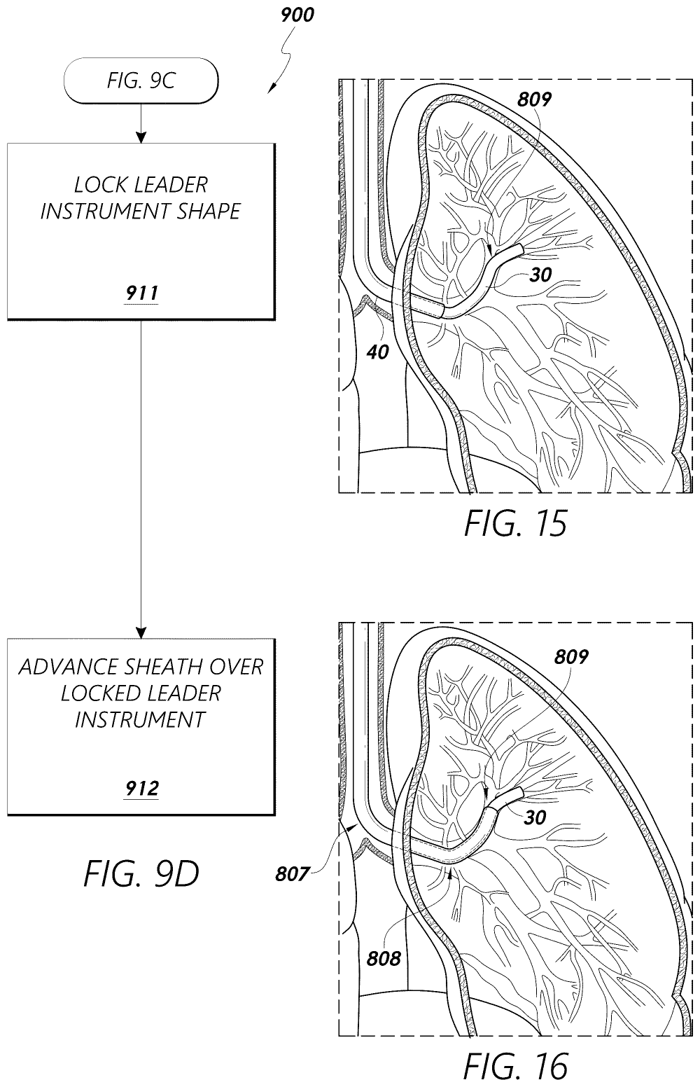

Shape-locking features disclosed herein can enable various multi-instrument driving processes that facilitate compound bending and navigation of tortuous anatomy. FIGS. 9A, 9B, 9C, and 9D provide a flow diagram illustrating a process 900 for implementing dual-instrument cooperative instrument advancement and articulation for navigating through complex anatomy in accordance with one or more embodiments. FIG. 10-16 show certain images corresponding to various blocks, states, and/or operations associated with the process 900 of FIGS. 9A, 9B, 9C, and 9D in accordance with one or more embodiments. Although illustrated in the context of pulmonary anatomy, it should be understood that the process 900 may be implemented in any suitable or desirable anatomy, such as within the gastrointestinal system of a subject. In particular, the process 900 may be particularly suitable for navigation in anatomy comprising tracks that are relatively long and tortuous, wherein navigation paths include turns in multiple different directions. For example, the process 900 can involve dynamically adjusting flexibility and stiffness/pushability of one or more instruments in an alternating manner to produce complex shapes in the instruments that are not achievable in the same manner using certain other solutions.

The process 900 can be implemented using two elongate instruments, such as a sheath 40 and an endoscope 30 disposed within a lumen of the sheath 40, wherein one or both of the instruments have pull wire tension locking feature(s) associated therewith to lock a present shape of the respective instrument. Either or both of the instruments 30, 40 may further be configured with intermediate pull wire fixation for a subset of the instrument's pull wires to provide force isolation as described in detail herein.

The process 900 is described below using a sheath-leader paradigm, which can be appropriate for robotic endoscopy procedures involving navigation of an endoscope and sheath in a cooperative manner in confined and complex anatomical pathways. In such paradigm, the endoscope 30 (or similar flexible instrument) is considered a leader instrument, and the sheath 40, which can be a tubular structure that covers the leader instrument. The process 900 involves sequential movement, wherein the leader instrument 30 paves the way and the sheath 40 provides support and passage for additional tools or interventions, providing a strategic, controlled advancement to navigate complex anatomical pathways safely and effectively.

At block 901, the process 900 involves advancing the leader instrument 30 and the sheath 40 to an anatomical junction, such as the area of the carina at the base of the trachea leading into the main bronchi of the pulmonary system, for example. FIG. 10 shows the leader instrument 30 and sheath 40 positioned in the respiratory anatomy of a patient 7 at a relevant junction 803.

Each of the leader instrument 30 and sheath 40 can include a plurality of pull wires having associated therewith tension locking features as described herein, such as wire clamps or pulley rotation locks, which can be configured to lock tension in some or all of the pull wires of a given instrument to create a stiffened member. By adjusting the state of the tension lock(s), the stiffness/flexibility of the shaft of the given instrument can be dynamically changed/controlled during the procedure/process 900. Furthermore, force-isolatable segments may be implemented that facilitate dynamically stiffening selected section(s) of the instrument shaft, such as by using a single motor to lock different sections of the shaft. For example, one or both of the instruments 30, 40 can be configured with multiple pull wire termination sections/plates as shown in FIG. 6A-6E and described above.

The process 900 can continue in a laddering process, wherein the sheath 40 is used as a stiff base support for advancement of the leader instrument 30, such that the leader instrument 30 is able to push-off of the sheath 40 to track deeper into the anatomy. For example, at block 902, the shape of the sheath 40 can be locked in accordance with aspects of the present disclosure to provide a stiff/rigid base for supporting distal advancement of the leader instrument 30.

At block 903, and illustrated in the image of FIG. 11, the process 900 involves driving the leader instrument 30 forward relative to the distal end of the sheath 40 and articulating the leader instrument 30 into a branch pathway 804. For example, when the leader instrument 30 reaches the anatomical junction 803, the articulation section (e.g., distal section) of the leader instrument 30 can be controlled so as to bank against the anatomy and continue forward. Such articulation of the leader instrument 30 can produce a bend 807 in the shaft thereof.

The leader instrument 30 and the sheath 40 can both be steerable, and can operate in a mother-daughter relationship, wherein both the leader instrument 30 and the sheath 40 can articulate independently. In some alternative implementations, the leader instrument may be steerable, whereas the sheath is not steerable or shape-locking and is elastic. In such implementations, the sheath is bent by the leader instrument rather than by its own articulation pull wires. Thus, higher articulation forces may be needed to articulate the leader instrument, which can have a negative impact on instrument profile/diameter requirements. In addition, once the leader instrument exits the sheath, the sheath may need to rely on anatomy to hold its shape, which is not always tenable and can cause stability issues. For implementations where both the leader instrument and the sheath are steerable, but where shape locking in accordance with aspects disclosed herein is not implemented, it may be necessary to have a very flexible distal end and stiff proximal end on both instruments. In certain scenarios, when the sheath is advanced relative to the leader instrument, the leader instrument may have a tendency to undesirably back-out of the anatomy to some degree due to prolapse. Therefore, dual steerable and shape-locking instruments as disclosed in detail herein can provide advantages over certain alternative solutions.

In some implementations, the sheath 40 may be co-articulated with the leader instrument 30 to counteract the forces imparted on the sheath 40 by the leader instrument 30. Alternatively, as shown in the process 900, the sheath 40 may passively counteract the forces of the leader instrument through sheath pull wire tension locking.

At block 904, the process 900 involves advancing the leader instrument 30 to a buckling limit or other desired stopping position, such as at a subsequent anatomical junction 805, as shown in the image of FIG. 12. For example, at some point, the leader instrument 30 may not be able to advance any further on its present path without an undesirable risk or certainty of buckling of the instrument. In some implementations, one or more sensors associated with the leader instrument 30 may provide signals indicating that movement of the instrument tip, or lack thereof, indicates buckling or risk of buckling from further advancement. The robotic system may detect the buckling state, or buckling risk state, and cease robotic advancement of the leader instrument 30 as a responsive action.

Once the limit position of the advanced leader instrument 30 has been reached, the process 900 may involve, at block 905, locking the shape of the leader instrument 30 in its articulated shape, including the bend 807. Further, at block 906, the previously-fixed/rigid shape of the sheath 40 may be unlocked. For example, while the rigid shape of the sheath can be helpful to provide support for advancement and articulation of the leader instrument 30, once the leader instrument 30 is articulated and shape-locked, such rigid support of the sheath 40 may not be needed for a temporary stage as the sheath is advanced to follow the leader instrument shape.

At block 907, the process 900 involves advancing the sheath over the locked shape of the leader instrument, as shown in the image of FIG. 13. With the shape of the leader instrument 30 in a sufficiently rigid locked state, passage of the sheath over the leader instrument 30 may advantageously not deform the previously-articulated shape of the leader instrument 30. Therefore, the sheath 40 can assume the shape of the leader instrument 30, including the bend 807 thereof.

Once the flexible unlocked sheath 40 has proceeded over the leader instrument 30, taking and copying its shape, with the distal end of the sheath at, past, or near the position of the distal end of the leader instrument 30, the shape of the sheath 40 may again be locked (block 908) and the leader instrument shape may be unlocked (block 909) to allow for further advancement of the presently flexible leader instrument 30.

At block 910, and shown in FIG. 14, the leader instrument 30 may be further advanced and articulated further into the anatomy. For example, the leader instrument 30 may be pushed forward down a further branch path 806, banking against anatomy until it reaches the next desired junction 811. The articulation of the leader instrument 30 can form a second bend 808 in the shaft thereof, such that the leader instrument 30, with the support of the sheath 40, includes multiple bends, which may or may not be in the same direction. In connection with the operation(s) of block 910, the articulation section (e.g., distal segment) of the shaft of the leader instrument 30 can bend to get around the junction 805. Further articulation of the leader instrument 30 can produce yet another bend 809 in the instrument shaft (see FIG. 15), such that the leader instrument can include three or more bends, allowing for navigation complexity requiring compound bending. The various bends 807, 808, 809 may be in any direction.

In connection with advancement and/or articulation of the leader instrument 30, at some point, buckling can once again occur or become a determined risk, such that it may be desirable or necessary for advancement of the leader instrument 30 to be halted. At such point, as shown at block 911, the leader instrument 30 may once again be shape-locked, after which the sheath 40 may (or may not) be unlocked and advanced over the compound bend shape of the locked leader instrument 30, in accordance with block 912 of the process 900.

The operations of any of blocks 901-912 may be repeated or omitted as desired to produce navigation and/or compound bending to accommodate the relevant procedure. The process 900 advantageously allows for the implementation of multiple (e.g., five or more) independent curvatures without the need for anatomical constraint

The process 900, as well as other processes and embodiments disclosed herein, can advantageously combine bend-and-stay instrument shape locking with articulation-based behavior in a manner as to provide optimal and/or dynamically-customizable instrument elasticity. For example, embodiments of the present disclosure allow for stiffness/flexibility adjustment in real time, such as by robotically controlling instruments to have different elasticity during dearticulation than during articulation. This can provide an additional layer of tuning to improve driving performance, such as by dampening-out jerk movements, and pull wire handoff with one-motor-per-plane articulation designs. Depending on the type of movement that the user is presently implementing, an appropriate level of dampening can be applied to address such movement type specifically. In addition, by allowing for the stiffening of one plane of articulation but not another, directional articulation accuracy can be improved in constrained environments.

The process 900 is described and shown in connection with procedures where an endoscope or other instrument is advanced from a distal opening of a sheath or other instrument, such that the endoscope and sheath cooperate in a telescoping relationship in at least some periods of the process. In some implementations, one or more additional instruments may be advanced within and/or relative to the endoscope and/or sheath to produce a telescoping arrangement in which three or more instruments (e.g., biopsy needle, endoscope, sheath, etc.) are positioned with distal ends thereof at three or more different axial/longitudinal positions. For example, with the endoscope 30 advanced beyond the distal end of the sheath 40, a biopsy needle or any other instrument or tool may be projected from a distal opening of a working channel of the endoscope 30 to produce a telescoping arrangement of at least three instruments. In such implementations, the most-distal instrument, in addition to one or more of the more-proximal instruments, may be steerable, such that multiple bends can be formed in the telescopic assembly, such as one or more bends at each of two or more axial segments of the assembly, each of such axial segments having a separate combination of instruments positioned in the segment.

### Additional Description of Examples and Terminology

Provided below is a list of examples, each of which may include aspects of any of the other examples disclosed herein. Furthermore, aspects of any example described above may be implemented in any of the numbered examples provided below.

Example 1: An instrument comprising a shaft, a first plurality of pull wire segments running a length of the shaft and terminating at a distal portion of the shaft, and a pull wire lock configured to lock tensions in the first plurality of pull wire segments.

Example 2: The instrument of any example herein, in particular example 1, further comprising a plurality of tubes running the length of the shaft, each of the first plurality of pull wire segments being disposed within one of the plurality of tubes.

Example 3: The instrument of any example herein, in particular example 2, further comprising a braided shaft associated with the shaft, wherein the plurality of tubes are interwoven with strands of the braided shaft.

Example 4: The instrument of any example herein, in particular example 3, further comprising an outer tube radially outside of the braided shaft, and an inner tube radially inside of the braided shaft.

Example 5: The instrument of any example herein, in particular example 2, wherein at least one of the plurality of tubes is usable as a working channel for tool, optical fiber, or electrical wire insertion through the instrument.

Example 6: The instrument of any example herein, in particular example 1, wherein the pull wire lock is configured to clamp down on the first plurality of pull wire segments proximal of the shaft.

Example 7: The instrument of any example herein, in particular example 1, wherein the pull wire lock is configured to transition between at least two tension-locking states including two or more of a free state in which the first plurality of pull wire segments are free to axially translate, a locked state in which the first plurality of pull wire segments are prevented from axially translating, or an intermediate resistance state in which the first plurality of pull wire segments are permitted to axially translate under increased resistance relative to the free state.

Example 8: The instrument of any example herein, in particular example 1, wherein the first plurality of pull wire segments are fixed to a first control plate positioned at the distal portion of the shaft, and the instrument further comprises a second plurality of pull wire segments fixed to a second control plate positioned between the distal portion of the shaft and a proximal end of the shaft.

Example 9: The instrument of any example herein, in particular example 8, wherein the first plurality of pull wire segments pass through the second control plate to the first control plate, and the first plurality of pull wire segments are not fixed to the second control plate.

Example 10: The instrument of any example herein, in particular example 8, wherein the pull wire lock is configured to lock the second plurality of pull wire segments independently of the first plurality of pull wire segments.

Example 11: The instrument of any example herein, in particular example 8, wherein the second control plate has a plurality of empty tubes passing therethrough.

Example 12: The instrument of any example herein, in particular example 1, further comprising a plurality of control plates distributed about the length of the shaft, wherein the instrument includes a plurality of shaft sections, each shaft section including one of the plurality of control plates and a proximally-adjacent segment of the shaft.

Example 13: The instrument of any example herein, in particular example 12, wherein two or more of the plurality of shaft sections have different lengths.

Example 14: A system comprising a sheath comprising an elongate tube and a first plurality of pull wire segments running a length of the elongate tube and terminating at a distal portion of the elongate tube. The system further comprises an endoscope configured to be disposed within a lumen of the elongate tube of the sheath, the endoscope comprising a shaft and a second plurality of pull wire segments running a length of the shaft and terminating at a distal portion of the shaft. The system further comprises a sheath pull wire lock configured to lock tensions in the first plurality of pull wire segments, and an endoscope pull wire lock configured to lock tension in the second plurality of pull wire segments.

Example 15: The system of any example herein, in particular example 14, wherein at least one of the sheath or the endoscope comprises multiple axially-offset sections, each of the multiple axially-offset sections having a separate set of pull wire segments fixed thereto.

Example 16: The system of any example herein, in particular example 14, wherein the sheath includes a braided jacket with a plurality of tubes interwoven therewith, and each of the first plurality of pull wire segments passes through one of the plurality of tubes.

Example 17: A method of any example herein, in particular navigating a medical instrument, the method comprising advancing a distal portion of an instrument from a distal end of a sheath, articulating the distal portion of the instrument to cause the distal portion of the instrument to assume a first shape, locking the distal portion of the instrument in the first shape, advancing a distal portion of the sheath over the distal portion of the instrument to cause the distal portion of the sheath to assume the first shape, and locking the distal portion of the sheath in the first shape.

Example 18: The method of any example herein, in particular example 17, wherein locking the distal portion of the sheath in the first shape involves clamping a plurality of pull wires associated with the sheath when the distal portion of the sheath is in the first shape.

Example 19: The method of any example herein, in particular example 17, further comprising, with the distal portion of the sheath locked in the first shape, unlocking the distal portion of the instrument, advancing the distal portion of the instrument from the distal end of the sheath, and articulating the distal portion of the instrument to cause the distal portion of the instrument to assume a second shape. The method further comprises locking the distal portion of the instrument in the second shape, advancing the distal portion of the sheath over the distal portion of the instrument to cause the distal portion of the sheath to assume the second shape, and locking the distal portion of the sheath in the second shape.

For purposes of summarizing the disclosure, certain aspects, advantages, and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim requires more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. An instrument comprising:
a shaft;
a first plurality of pull wire segments running a length of the shaft and terminating at a distal portion of the shaft; and
a pull wire lock configured to lock tensions in the first plurality of pull wire segments.

2. The instrument of claim 1, further comprising a plurality of tubes running the length of the shaft, each of the first plurality of pull wire segments being disposed within one of the plurality of tubes.

3. The instrument of claim 2, further comprising a braided shaft associated with the shaft, wherein the plurality of tubes are interwoven with strands of the braided shaft.

4. The instrument of claim 3, further comprising:
an outer tube radially outside of the braided shaft; and
an inner tube radially inside of the braided shaft.

5. The instrument of claim 2, wherein at least one of the plurality of tubes is usable as a working channel for tool, optical fiber, or electrical wire insertion through the instrument.

6. The instrument of claim 1, wherein the pull wire lock is configured to clamp down on the first plurality of pull wire segments proximal of the shaft.

7. The instrument of claim 1, wherein the pull wire lock is configured to transition between at least two tension-locking states including two or more of:
a free state in which the first plurality of pull wire segments are free to axially translate;
a locked state in which the first plurality of pull wire segments are prevented from axially translating; or
an intermediate resistance state in which the first plurality of pull wire segments are permitted to axially translate under increased resistance relative to the free state.

8. The instrument of claim 1, wherein:
the first plurality of pull wire segments are fixed to a first control plate positioned at the distal portion of the shaft; and
the instrument further comprises a second plurality of pull wire segments fixed to a second control plate positioned between the distal portion of the shaft and a proximal end of the shaft.

9. The instrument of claim 8, wherein:
the first plurality of pull wire segments pass through the second control plate to the first control plate; and
the first plurality of pull wire segments are not fixed to the second control plate.

10. The instrument of claim 8, wherein the pull wire lock is configured to lock the second plurality of pull wire segments independently of the first plurality of pull wire segments.

11. The instrument of claim 8, wherein the second control plate has a plurality of empty tubes passing therethrough.

12. The instrument of claim 1, further comprising a plurality of control plates distributed about the length of the shaft, wherein the instrument includes a plurality of shaft sections, each shaft section including one of the plurality of control plates and a proximally-adjacent segment of the shaft.

13. The instrument of claim 12, wherein two or more of the plurality of shaft sections have different lengths.

14. A system comprising:
a sheath comprising:
an elongate tube; and
a first plurality of pull wire segments running a length of the elongate tube and terminating at a distal portion of the elongate tube;
an endoscope configured to be disposed within a lumen of the elongate tube of the sheath, the endoscope comprising:
a shaft; and
a second plurality of pull wire segments running a length of the shaft and terminating at a distal portion of the shaft;
a sheath pull wire lock configured to lock tensions in the first plurality of pull wire segments; and
an endoscope pull wire lock configured to lock tension in the second plurality of pull wire segments.

15. The system of claim 14, wherein at least one of the sheath or the endoscope comprises multiple axially-offset sections, each of the multiple axially-offset sections having a separate set of pull wire segments fixed thereto.

16. The system of claim 14, wherein:
the sheath includes a braided jacket with a plurality of tubes interwoven therewith; and
each of the first plurality of pull wire segments passes through one of the plurality of tubes.

17. A method of navigating a medical instrument, the method comprising:
advancing a distal portion of an instrument from a distal end of a sheath;
articulating the distal portion of the instrument to cause the distal portion of the instrument to assume a first shape;
locking the distal portion of the instrument in the first shape;
advancing a distal portion of the sheath over the distal portion of the instrument to cause the distal portion of the sheath to assume the first shape; and
locking the distal portion of the sheath in the first shape.

18. The method of claim 17, wherein locking the distal portion of the sheath in the first shape involves clamping a plurality of pull wires associated with the sheath when the distal portion of the sheath is in the first shape.

19. The method of claim 17, further comprising:
with the distal portion of the sheath locked in the first shape:
unlocking the distal portion of the instrument;
advancing the distal portion of the instrument from the distal end of the sheath; and
articulating the distal portion of the instrument to cause the distal portion of the instrument to assume a second shape;
locking the distal portion of the instrument in the second shape;
advancing the distal portion of the sheath over the distal portion of the instrument to cause the distal portion of the sheath to assume the second shape; and
locking the distal portion of the sheath in the second shape.
